(19) United States
(12) Patent Application Publication
(10) Pub. No.: US 2017/0333319 A1
(43) Pub. Date: Nov. 23, 2017

(54) DEODORIZING COMBINATION OF ACTIVE INGREDIENTS

(71) Applicant: Henkel AG & Co. KGaA, Duesseldorf (DE)

(72) Inventors: Thomas Doering, Dormagen (DE); Natascha Schevardo, Erkrath (DE)

(73) Assignee: Henkel AG & Co. KGaA, Duesseldorf (DE)

(21) Appl. No.: 15/599,224

(22) Filed: May 18, 2017

(30) Foreign Application Priority Data
May 23, 2016 (DE) 102016208858.0

(51) Int. Cl. A61K 8/41; A61K 8/34; A61Q 15/00

(52) U.S. Cl. CPC A61K 8/416; A61Q 15/00; A61K 8/347; A61K 8/34; A61K 8/345; A61K 2800/30

(57) ABSTRACT

A deodorizing cosmetic agent in the form of an aqueous emulsion which includes:
at least one quaternary ammonium compound and
ethylhexyl glycerol,
   wherein the deodorizing cosmetic agent does not include any aluminum-containing compounds, use and methods associated therewith, using such an agent.

### FIELD OF THE INVENTION

The present invention generally relates to cosmetic deodorant compositions which have an improved effect, and methods when using these compositions.

### BACKGROUND OF THE INVENTION

In many cultural groups, cleaning and care of the body includes measures which are intended to counteract the onset of an intense body odor which is considered to be unpleasant. The onset of body odor is facilitated inter alia by transpiration, which is why the cosmetic industry provides numerous toiletries which have been developed for application in regions of the body with a high density of sweat glands, in particular in the armpit area. These toiletries have a perspiration-inhibiting (antiperspirant) and/or deodorizing (deodorant) effect.

In addition to at least one oil or a fatty substance and an odoriferous component or a perfume, cosmetic antiperspirants of the prior art usually include at least one perspiration-inhibiting salt. On the one hand, the perspiration-inhibiting salts used in these antiperspirants reduce sweat secretion by the body by temporarily narrowing and/or blocking the excretory ducts of the sweat glands, with the result that the quantity of sweat can be reduced by approximately 20 to 60 percent. On the other hand, because of their antimicrobial effect, they display an additional deodorizing effect. Activated basic aluminum and aluminum-zirconium halogenides are commonly used as perspiration-inhibiting salts. Furthermore, aluminum and aluminum-zirconium halogenides which are stabilized with organic acids as complex ligands can also be used. People with sensitive skin can react to perspiration-inhibiting salts with skin irritations. Aluminum-free antiperspirants and deodorants are described for example in German patent applications DE102013225617 A1 and DE102013225620 A1.

Antiperspirants and deodorants can be packaged in the most varied administration forms, for example as a powder, in the form of a pencil, as an aerosol spray, as a liquid and gel-like roll-on application, cream, gel and as impregnated flexible substrates (deodorant wipes).

Packaging the antiperspirants and deodorants in liquid form has advantages in respect of dosing and application, for example by means of a nebulizer or spray. Low-viscosity emulsion systems suitable for nebulization however do not have sufficient storage stability.

### BRIEF SUMMARY OF THE INVENTION

It has now been surprisingly found that the aforementioned technical problems are solved by aluminum-free compositions which include at least one quaternary ammonium compound in combination with ethylhexyl glycerol. The present invention provides:

1. A deodorizing cosmetic agent in the form of an aqueous emulsion which includes:
at least one quaternary ammonium compound and
ethylhexyl glycerol,
   wherein the deodorizing cosmetic agent does not include any aluminum-containing compounds.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has now been surprisingly found that the aforementioned technical problems are solved by aluminum-free compositions which include at least one quaternary ammonium compound in combination with ethylhexyl glycerol. The present invention provides:

A deodorizing cosmetic agent in the form of an aqueous emulsion which includes: at least one quaternary ammonium compound and ethylhexyl glycerol, wherein the deodorizing cosmetic agent does not include any aluminum-containing compounds.

The deodorizing cosmetic agents according to point 1, wherein the agent includes at least one compound, selected from the group of behenyl trimonium chloride, cetrimonium bromide, cetrimonium chloride, lauryl trimonium bromide, lauryl trimonium chloride, stearyl trimonium bromide, stearyl trimonium chloride, benzethonium chloride, benzalkonium chloride as well as their mixtures, as quaternary ammonium compound.

The deodorizing cosmetic agents according to one of the preceding points, wherein the agent includes benzalkonium chloride as quaternary ammonium compound.

The deodorizing cosmetic agent according to one of the preceding points, wherein the agent includes the quaternary ammonium compound in a quantity of from 0.01 to 0.3 wt.-%, preferably of from 0.02 to 0.2 wt.-% and in particular of from 0.05 to 0.1 wt.-%, relative to the total weight of the agent.

The deodorizing cosmetic agent according to one of the preceding points, wherein the agent includes the ethylhexyl glycerol in a quantity of from 0.1 to 3.0 wt.-%, preferably of from 0.15 to 2.0 wt.-% and in particular of from 0.2 to 1.0 wt.-%, relative to the total weight of the agent.

The deodorizing cosmetic agent according to one of the preceding points, wherein the agent includes at least one bactericide from the group of chlorhexidine, farnesol, triclosan, chloroxylenol and phenoxy ethanol, preferably phenoxy ethanol.

The deodorizing cosmetic agent according to one of the preceding points, wherein the agent includes 0.1 to 2.0 wt.-%, preferably 0.2 to 1.6 wt.-% and in particular 0.5 to 1.4 wt.-% of a bactericide, preferably phenoxy ethanol.

The deodorizing cosmetic agent according to one of the preceding points, wherein the agent includes at least one oil, liquid at 20° C. and 1.013 hPa, from the group of
esters of linear or branched saturated or unsaturated C2-30 fatty alcohols with linear or branched saturated or unsaturated C2-30 fatty acids,
products of addition of ethylene oxide and/or propylene oxide to mono- or polyvalent C3-22 alkanols
dialkyl ether and
paraffins.

The deodorizing cosmetic agent according to one of the preceding points, wherein the agent includes at least one oil, liquid at 20° C. and 1.013 hPa, in a quantity of from 0.1 to 15 wt.-%, preferably of from 0.2 to 10 wt.-% and in particular of from 0.5 to 5.0 wt.-%, relative to the total weight of the agent.

The deodorizing cosmetic agent according to one of the preceding points, wherein the agent includes at least one non-ionic emulsifier from the group of products of addition of 2 to 50 mol ethylene oxide to linear fatty alcohols with 12 to 18 carbon atoms and of products of addition of 2 to 50 mol propylene oxide to linear fatty alcohols with 12 to 18 carbon atoms.

The deodorizing cosmetic agent according to point 10, wherein, relative to the total weight of the agent, the agent includes the non-ionic emulsifier in a quantity of from 3.0 to 12 wt.-%, preferably of from 4.0 to 10 wt.-% and in particular of from 5.0 to 8.0 wt.-%.

The deodorizing cosmetic agent according to one of the preceding points, wherein the agent includes ethanol in quantities of less than 5.0 wt.-%, preferably less than 1.0 wt.-% and in particular less than 0.1 wt.-%, relative to the total weight of the agent.

The deodorizing cosmetic agent according to one of the preceding points, wherein the agent is free from ethanol.

The deodorizing cosmetic agent according to one of the preceding points, wherein the agent is present as an oil-in-water emulsion.

The deodorizing cosmetic agent according to one of the preceding points, wherein the agent includes water in quantities of 75 to 96 wt.-%, preferably 80 to 96 wt.-% and in particular 85 to 94 wt.-%, relative to the total weight of the agent.

The deodorizing cosmetic agent according to one of the preceding points, wherein the agent is free from propellant.

A pump nebulizer containing a deodorizing cosmetic agent according to one of the preceding points.

A roll-on applicator containing a deodorizing cosmetic agent according to one of the preceding points.

A use of a deodorizing cosmetic agent according to one of the preceding points, for preventing and/or reducing body odor.

A non-therapeutic cosmetic method for preventing and/or reducing body odor, in which a cosmetic agent according to one of the preceding points is applied to the skin, in particular to the skin of the armpits, and remains on the skin for at least 1 hour, preferably for at least 2 hours, preferably for at least 4 hours, in particular for at least 6 hours.

The cosmetic agents according to the invention include, as their first essential constituent, at least one quaternary ammonium compound. With regard to their technical properties, in particular their storage stability and cosmetic effect, cosmetic agents which include at least one compound selected from the group of behenyl trimonium chloride, cetrimonium bromide, cetrimonium chloride, lauryl trimonium bromide, lauryl trimonium chloride, stearyl trimonium bromide, stearyl trimonium chloride, benzethonium chloride and benzalkonium chloride, as quaternary ammonium compound, have proved advantageous. The use of mixtures of the aforementioned quaternary ammonium compounds is of course also possible.

A technically particularly preferred quaternary ammonium compound is benzalkonium chloride.

The percentage by weight of quaternary ammonium compounds in the total weight of the cosmetic agent is preferably of from 0.01 to 0.3 wt.-%, preferably of from 0.02 to 0.2 wt.-% and in particular of from 0.05 to 0.1 wt.-%.

The second essential constituent of the cosmetic agents according to the invention is ethylhexyl glycerol. The percentage by weight of ethylhexyl glycerol in the total weight of the cosmetic agent is preferably of from 0.1 to 3.0 wt.-%, preferably of from 0.15 to 2.0 wt.-% and in particular of from 0.2 to 1.0 wt.-%.

The cosmetic agents are present as aqueous emulsions. Preferred cosmetic agents are characterized by a water content of from 75 to 96 wt.-%, preferably 80 to 96 wt.-% and in particular 85 to 94 wt.-% (in each case relative to the total weight of the agent).

The cosmetic agents do not include any aluminum-containing compounds. Within the framework of the present invention, the term “aluminum-containing compounds” is understood to mean perspiration-inhibiting aluminum salts and aluminum-zirconium salts.

Cosmetic deodorants counteract, cover or eliminate body odors. Body odors are produced by the effect of skin bacteria on apocrine sweat, wherein disagreeable-smelling degradation products are formed. Accordingly, preferred deodorants include active ingredients which function as germ-inhibiting agents, enzyme inhibitors, odor absorbers or odor masks.

In principle, all substances which are effective against gram-positive bacteria, such as e.g. 4-hydroxybenzoic acid and its salts and esters, N-(4-chlorophenyl)-N′-(3,4 dichlorophenyl)urea, 2,4,4′-trichloro-2′-hydroxydiphenyl ether (triclosan), 4-chloro-3,5-dimethylphenol, 2,2′-methylen-bis(6-bromo-4-chlorophenol), 3-methyl-4-(1-methyl ethyl)phenol, 2-benzyl-4-chlorophenol, 3-(4-chlorophenoxy)-1,2-propane diol, 3-iodo-2-propynyl butylcarbamate, chlorhexidine, 3,4,4′-trichlorocarbanilide (TTC), antibacterial scents, thymol, thyme oil, eugenol, clove oil, mint oil, farnesol, phenoxy ethanol, glycerol monolaurate (GML), diglycerol monocaprylate (DMC), salicylic acid-N-alkylamides such as e.g. salicylic acid-n-octylamide or salicylic acid-n-decylamide, are suitable as germ-inhibiting agents which can optionally be added to the cosmetic agents according to the invention.

Enzyme inhibitors can be added to the cosmetic agents according to the invention, provided that they do not inhibit the enzymatic activities which bring about biocide activation or release in the presence of the microorganisms being targeted. By complying with this condition, for example esterase inhibitors are possible suitable enzyme inhibitors. These are preferably trialkyl citrates such as trimethyl citrate, tripropyl citrate, triisopropyl citrate, tributyl citrate and in particular triethyl citrate (Hydagen® CAT, Henkel KGaA, Düsseldorf/FRG). The substances inhibit enzyme activity and thereby reduce the onset of odor. Further substances which can be considered as esterase inhibitors are sterol sulfates or phosphates, such as for example lanosterol sulfate or phosphate, cholesterol sulfate or phosphate, campesterol sulfate or phosphate, stigmasterol sulfate or phosphate and sitosterol sulfate or phosphate, dicarboxylic acids and their esters, such as for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and their esters, such as for example citric acid, malic acid, tartaric acid or tartaric acid diethyl ester, and zinc glycinate.

In combination with the combination of active ingredients according to the invention, the use of bactericides has proven particularly advantageous. Their use, in particular the use of a bactericide from the group of chlorhexidine, farnesol, triclosan, chloroxylenol and phenoxy ethanol, preferably phenoxy ethanol, had an unexpected advantageous influence on the formation of the odor of the cosmetic agents. Deodorizing cosmetic agents which include a bactericide from the group of chlorhexidine, farnesol, triclosan, chloroxylenol and phenoxy ethanol, preferably phenoxy ethanol, are therefore preferred.

The percentage by weight of the bactericide, preferably phenoxy ethanol, in the total weight of the cosmetic agent is preferably of from 0.1 to 2.0 wt.-%, preferably of from 0.2 to 1.6 wt.-% and in particular of from 0.5 to 1.4 wt.-%.

In addition to the aforementioned active substances the cosmetic agents can include further active ingredients and auxiliaries. A first group of preferred cosmetic active ingredients are oils (cosmetic oils).

The term “cosmetic oil” is understood to mean an oil suitable for cosmetic use which cannot be mixed with water. The cosmetic oils used according to the invention are neither scents nor essential oils. Deodorizing cosmetic agents which include at least one oil liquid at 20° C. and 1.013 hPa are preferred according to the invention.

According to a particularly preferred embodiment, the cosmetic oil liquid at 20° C. and 1.013 hPa is selected from the group of
(i) volatile cyclical silicone oils, in particular cyclotrisiloxane, cyclotetrasiloxane, cyclopentasiloxane and cyclohexasiloxane, and linear silicone oils with 2 to 10 siloxane units, in particular hexamethyldisiloxane, octamethyltrisiloxane, decamethyltetrasiloxane;
(ii) volatile non-silicone oils, in particular liquid paraffin oils and isoparaffin oils, such as isodecane, isoundecane, isododecane, isotridecane, isotetradecane, isopentadecane, isohexadecane and isoeicosane;
(iii) non-volatile silicone oils, in particular high-molecular linear polyalkyl siloxanes;
(iv) non-volatile non-silicone oils, in particular esters of linear or branched saturated or unsaturated C₂₋₃₀ fatty alcohols having linear or branched saturated or unsaturated C₂₋₃₀ fatty acids, which can be hydroxylated, C₈-C₂₂ fatty alcohol esters of monovalent or polyvalent C₂-C₇ hydroxy carboxylic acids, triethyl citrates, branched saturated or unsaturated C₆₋₃₀ fatty alcohols, mono-, di- and triglycerides of linear or branched, saturated or unsaturated, optionally hydroxylated C₈₋₃₀ fatty acids, dicarboxylic acid esters of linear or branched C₂-C₁₀ alkanols, products of addition of ethylene oxide and/or propylene oxide to mono- or polyvalent C₃₋₂₂ alkanols, which can optionally be esterified, symmetrical, asymmetrical or cyclical esters of carbonic acid with fatty alcohols, esters of dimers of unsaturated C₁₂₋₂₂ fatty acids having monovalent, linear, branched and cyclical C₂₋₁₈ alkanols or C₂₋₆ alkanols, benzoic acid esters of linear or branched C₈₋₂₂ alkanols, such as benzoic acid C₁₂₋₁₅ alkyl ester and benzoic acid isostearyl ester and benzoic acid octyldodecyl ester, synthetic hydrocarbons such as polyisobutene and polydecene, alicyclic hydrocarbons;
(v) mixtures thereof.

The term “volatile cosmetic oil” denotes cosmetic oils which, at 20° C. and an ambient pressure of 1.013 hPa, have a vapor pressure of from 2.66 Pa to 40,000 Pa (0.02 to 300 mm Hg), preferably of from 10 to 12,000 Pa (0.1 to 90 mm Hg), more preferably of from 13 to 3,000 Pa (0.1 to 23 mm Hg) and in particular of from 15 to 500 Pa (0.1 to 4 mm Hg).

The term “non-volatile cosmetic oils” is understood to mean cosmetic oils which, at 20° C. and an ambient pressure of 1.013 hPa, have a vapor pressure of less than 2.66 Pa (0.02 mm Hg).

Particularly preferred cosmetic agents include at least one oil liquid at 20° C. and 1.013 hPa from the group of
esters of linear or branched saturated or unsaturated C2-30 fatty alcohols having linear or branched saturated or unsaturated C2-30 fatty acids,
products of addition of ethylene oxide and/or propylene oxide to mono- or polyvalent C3-22 alkanols
dialkyl ether and
paraffins.
   In conjunction with the active ingredient combination, according to the invention, of quaternary ammonium compound and hydroxyacetophenone, these oils have proven particularly effective.

Relative to their total weight, preferred deodorizing cosmetic agents include oil in quantities of from 0.1 to 15 wt.-%, preferably of from 0.2 to 10 wt.-% and in particular of from 0.5 to 5.0 wt.-%.

Preferred cosmetic agents are present as oil-in-water emulsions.

The agents according to the invention include at least one emulsifier as further preferred constituent. In principle, anionic, cationic, non-ionic and ampholytic surface active compounds are considered as emulsifiers which are suitable for application to the human body. The ampholytic surface active compounds comprise zwitterionic surface active compounds and ampholytes. Non-ionic emulsifiers are preferred.

In particular, products of addition of ethylene oxide to linear fatty alcohols, to fatty acids, to fatty acid alkanolamides, to fatty acid monoglycerides, to sorbitan fatty acid monoesters, to fatty acid glycerides, to methylglucoside mono fatty acid esters, to polydimethylsiloxanes and mixtures thereof, can be used as non-ionic emulsifiers.

The emulsifier is preferably selected from the group of products of addition of 2 to 50 mol ethylene oxide to linear fatty alcohols having 12 to 18 carbon atoms and of products of addition of 2 to 50 mol propylene oxide to linear fatty alcohols having 12 to 18 carbon atoms. Examples of particularly preferred non-ionic surfactants are compounds with the INCI names steareth-2, steareth-21 and in particular their mixtures.

Preferred deodorizing cosmetic agents include the non-ionic emulsifier in a quantity of from 3.0 to 12 wt.-%, preferably of from 4.0 to 10 wt.-% and in particular of from 5.0 to 8.0 wt.-%, relative to the total weight of the agent.

Furthermore, the cosmetic agents can include at least one chelating agent which is preferably selected from ethylenediaminetetraacetic acid (EDTA) and its salts as well as from nitrilotriacetic acid (NTA) and mixtures of these substances, in a total quantity of from 0.01 to 0.5 wt.-%, preferably of from 0.02 to 0.3 wt.-%, in particular of from 0.05 to 0.2 wt.-%, relative to the total weight of the cosmetic agent.

As stated at the outset, the alcohol content of deodorizing cosmetic agents can have a disadvantageous effect on their skin compatibility. The combination of active ingredients according to the invention makes possible the provision of effective deodorants with a reduced alcohol content. To improve skin compatibility it is therefore preferred that, relative to their total weight, the deodorizing cosmetic agents include ethanol in quantities of less than 5.0 wt.-%, preferably less than 1.0 wt.-% and in particular less than 0.1 wt.-%. It is particularly preferred if the agent is free from ethanol.

In addition to the aforementioned ingredients, the deodorizing cosmetic agents can include further active ingredients and auxiliaries. This group of further active ingredients and auxiliaries includes for example scents.

Particularly preferred cosmetic agents according to the invention also include at least one scent. The term “scent” denotes a chemical compound with an odor and/or aroma which stimulates the receptors of the hair cells of the olfactory system (appropriate irritation). The physical and chemical properties necessary for this are a low molecular mass of at most 300 g/mol, a high vapor pressure, minimal water solubility and high fat solubility as well as weak polarity and the presence of at least one osmophore in the molecule. In order to differentiate volatile, low-molecular substances which are conventionally and also within the meaning of the present invention considered and used, not as a scent but principally as a solvent, such as for example ethanol, propanol, isopropanol and acetone, from scents, the scents according to the invention have a molecular mass of from 74 to 300 g/mol, include at least one osmophore in the molecule and have an odor and/or aroma, i.e. they stimulate the receptors of the hair calls of the olfactory system.

Perfumes, perfume oils or constituents of perfume oils can be used as scents. According to the invention, perfume oils or fragrances can be individual scent compounds, e.g. the synthetic products of esters, ethers, aldehydes, ketones, alcohols and hydrocarbons. Ester-type scent compounds are e.g. benzyl acetate, phenoxyethyl isobutyrate, p-tert-butyl cyclohexyl acetate, linalyl acetate, dimethyl benzyl carbinyl acetate (DMBCA), phenyl ethyl acetate, benzyl acetate, ethyl methyl phenyl glycinate, allyl cyclohexyl propionate, styrallyl propionate, benzyl salicylate, cyclohexyl salicylate, floramate, melusate and jasmecyclate. The ethers include for example benzyl ethyl ether and ambroxan, the aldehydes include e.g. linear alkanals having 8-18 C atoms, citral, citronellal, citronellyloxy acetaldehyde, cyclamen aldehyde, lilial and bourgeonal, the ketones include e.g. ionone, alpha-isomethyl ionone and methyl cedryl ketone, the alcohols include anethole, citronellol, eugenol, geraniol, linalool, phenylethyl alcohol and terpineol, the hydrocarbons include mainly terpenes such as limonene and pinene. However, mixtures of different scents are preferably used which together produce an appealing fragrance. Preferred cosmetic agents according to the invention include at least one fragrance component in an overall quantity of from 0.00001 to 10 wt.-%, preferably 0.5 to 5 wt.-%, exceptionally preferably 1 to 4 wt.-%, relative in each case to the total weight of the agent.

The composition of some preferred cosmetic agents can be seen in the following tables (details in wt.-% relative to the total weight of the cosmetic agent unless otherwise indicated).

| | Formula 1 | Formula 2 | Formula 3 | Formula 4 | Formula 5 |
|---|---|---|---|---|---|
| Quaternary ammonium | 0.01 to 0.3 | 0.01 to 0.3 | 0.02 to 0.2 | 0.02 to 0.2 | 0.05 to 0.1 |
| compound | | | | | |
| Ethylhexyl glycerol | 0.1 to 3.0 | 0.15 to 2.0 | 0.15 to 2.0 | 0.2 to 1.0 | 0.2 to 1.0 |
| Water, optional | up to 100 | up to 100 | up to 100 | up to 100 | up to 100 |
| additive | | | | | |
| | | | | | Formula |
| | Formula 6 | Formula 7 | Formula 8 | Formula 9 | 10 |
| Benzalkonium chloride | 0.01 to 0.3 | 0.01 to 0.3 | 0.02 to 0.2 | 0.02 to 0.2 | 0.05 to 0.1 |
| Ethylhexyl glycerol | 0.1 to 3.0 | 0.15 to 2.0 | 0.15 to 2.0 | 0.2 to 1.0 | 0.2 to 1.0 |
| Water, optional | up to 100 | up to 100 | up to 100 | up to 100 | up to 100 |
| additive | | | | | |
| | Formula | Formula | Formula | Formula | Formula |
| | 11 | 12 | 13 | 14 | 15 |
| Quaternary ammonium | 0.01 to 0.3 | 0.01 to 0.3 | 0.02 to 0.2 | 0.02 to 0.2 | 0.05 to 0.1 |
| compound | | | | | |
| Ethylhexyl glycerol | 0.1 to 3.0 | 0.15 to 2.0 | 0.15 to 2.0 | 0.2 to 1.0 | 0.2 to 1.0 |
| Bactericide | 0.1 to 2.0 | 0.1 to 2.0 | 0.2 to 1.6 | 0.2 to 1.6 | 0.5 to 1.4 |
| Water, optional | up to 100 | up to 100 | up to 100 | up to 100 | up to 100 |
| additive | | | | | |
| | Formula | Formula | Formula | Formula | Formula |
| | 16 | 17 | 18 | 19 | 20 |
| Benzalkonium chloride | 0.01 to 0.3 | 0.01 to 0.3 | 0.02 to 0.2 | 0.02 to 0.2 | 0.05 to 0.1 |
| Ethylhexyl glycerol | 0.1 to 3.0 | 0.15 to 2.0 | 0.15 to 2.0 | 0.2 to 1.0 | 0.2 to 1.0 |
| Bactericide | 0.1 to 2.0 | 0.1 to 2.0 | 0.2 to 1.6 | 0.2 to 1.6 | 0.5 to 1.4 |
| Water, optional | up to 100 | up to 100 | up to 100 | up to 100 | up to 100 |
| additive | | | | | |
| | Formula | Formula | Formula | Formula | Formula |
| | 21 | 22 | 23 | 24 | 25 |
| Quaternary ammonium | 0.01 to 0.3 | 0.01 to 0.3 | 0.02 to 0.2 | 0.02 to 0.2 | 0.05 to 0.1 |
| compound | | | | | |
| Ethylhexyl glycerol | 0.1 to 3.0 | 0.15 to 2.0 | 0.15 to 2.0 | 0.2 to 1.0 | 0.2 to 1.0 |
| Phenoxy ethanol | 0.1 to 2.0 | 0.1 to 2.0 | 0.2 to 1.6 | 0.2 to 1.6 | 0.5 to 1.4 |
| Water, optional | up to 100 | up to 100 | up to 100 | up to 100 | up to 100 |
| additive | | | | | |
| | Formula | Formula | Formula | Formula | Formula |
| | 26 | 27 | 28 | 29 | 30 |
| Benzalkonium chloride | 0.01 to 0.3 | 0.01 to 0.3 | 0.02 to 0.2 | 0.02 to 0.2 | 0.05 to 0.1 |
| Ethylhexyl glycerol | 0.1 to 3.0 | 0.15 to 2.0 | 0.15 to 2.0 | 0.2 to 1.0 | 0.2 to 1.0 |
| Phenoxy ethanol | 0.1 to 2.0 | 0.1 to 2.0 | 0.2 to 1.6 | 0.2 to 1.6 | 0.5 to 1.4 |
| Water, optional | up to 100 | up to 100 | up to 100 | up to 100 | up to 100 |
| additive | | | | | |
| | Formula | Formula | Formula | Formula | Formula |
| | 31 | 32 | 33 | 34 | 35 |
| Quaternary ammonium | 0.01 to 0.3 | 0.01 to 0.3 | 0.02 to 0.2 | 0.02 to 0.2 | 0.05 to 0.1 |
| compound | | | | | |
| Ethylhexyl glycerol | 0.1 to 3.0 | 0.15 to 2.0 | 0.15 to 2.0 | 0.2 to 1.0 | 0.2 to 1.0 |
| Non-ionic emulsifier | 3.0 to 12 | 3.0 to 12 | 4.0 to 10 | 4.0 to 10 | 5.0 to 8.0 |
| Water, optional | up to 100 | up to 100 | up to 100 | up to 100 | up to 100 |
| additive | | | | | |
| | Formula | Formula | Formula | Formula | Formula |
| | 36 | 37 | 38 | 39 | 40 |
| Benzalkonium chloride | 0.01 to 0.3 | 0.01 to 0.3 | 0.02 to 0.2 | 0.02 to 0.2 | 0.05 to 0.1 |
| Ethylhexyl glycerol | 0.1 to 3.0 | 0.15 to 2.0 | 0.15 to 2.0 | 0.2 to 1.0 | 0.2 to 1.0 |
| Non-ionic emulsifier | 3.0 to 12 | 3.0 to 12 | 4.0 to 10 | 4.0 to 10 | 5.0 to 8.0 |
| Water, optional | up to 100 | up to 100 | up to 100 | up to 100 | up to 100 |
| additive | | | | | |
| | Formula | Formula | Formula | Formula | Formula |
| | 41 | 42 | 43 | 44 | 45 |
| Quaternary ammonium | 0.01 to 0.3 | 0.01 to 0.3 | 0.02 to 0.2 | 0.02 to 0.2 | 0.05 to 0.1 |
| compound | | | | | |
| Ethylhexyl glycerol | 0.1 to 3.0 | 0.15 to 2.0 | 0.15 to 2.0 | 0.2 to 1.0 | 0.2 to 1.0 |
| Non-ionic emulsifier* | 3.0 to 12 | 3.0 to 12 | 4.0 to 10 | 4.0 to 10 | 5.0 to 8.0 |
| Water, optional | up to 100 | up to 100 | up to 100 | up to 100 | up to 100 |
| additive | | | | | |

*Steareth-2, steareth-21 or their combinations

| | Formula | Formula | Formula | Formula | Formula |
|---|---|---|---|---|---|
| | 46 | 47 | 48 | 49 | 50 |
| Benzalkonium chloride | 0.01 to 0.3 | 0.01 to 0.3 | 0.02 to 0.2 | 0.02 to 0.2 | 0.05 to 0.1 |
| Ethylhexyl glycerol | 0.1 to 3.0 | 0.15 to 2.0 | 0.15 to 2.0 | 0.2 to 1.0 | 0.2 to 1.0 |
| Non-ionic emulsifier* | 3.0 to 12 | 3.0 to 12 | 4.0 to 10 | 4.0 to 10 | 5.0 to 8.0 |
| Water, optional | up to 100 | up to 100 | up to 100 | up to 100 | up to 100 |
| additive | | | | | |

*Steareth-2, steareth-21 or combinations thereof

| | Formula | Formula | Formula | Formula | Formula |
|---|---|---|---|---|---|
| | 51 | 52 | 53 | 54 | 55 |
| Quaternary ammonium | 0.01 to 0.3 | 0.01 to 0.3 | 0.02 to 0.2 | 0.02 to 0.2 | 0.05 to 0.1 |
| compound | | | | | |
| Ethylhexyl glycerol | 0.1 to 3.0 | 0.15 to 2.0 | 0.15 to 2.0 | 0.2 to 1.0 | 0.2 to 1.0 |
| Bactericide | 0.1 to 2.0 | 0.1 to 2.0 | 0.2 to 1.6 | 0.2 to 1.6 | 0.5 to 1.4 |
| Non-ionic emulsifier | 3.0 to 12 | 3.0 to 12 | 4.0 to 10 | 4.0 to 10 | 5.0 to 8.0 |
| Water, optional | up to 100 | up to 100 | up to 100 | up to 100 | up to 100 |
| additive | | | | | |
| | Formula | Formula | Formula | Formula | Formula |
| | 56 | 57 | 58 | 59 | 60 |
| Benzalkonium chloride | 0.01 to 0.3 | 0.01 to 0.3 | 0.02 to 0.2 | 0.02 to 0.2 | 0.05 to 0.1 |
| Ethylhexyl glycerol | 0.1 to 3.0 | 0.15 to 2.0 | 0.15 to 2.0 | 0.2 to 1.0 | 0.2 to 1.0 |
| Bactericide | 0.1 to 2.0 | 0.1 to 2.0 | 0.2 to 1.6 | 0.2 to 1.6 | 0.5 to 1.4 |
| Non-ionic emulsifier | 3.0 to 12 | 3.0 to 12 | 4.0 to 10 | 4.0 to 10 | 5.0 to 8.0 |
| Water, optional | up to 100 | up to 100 | up to 100 | up to 100 | up to 100 |
| additive | | | | | |
| | Formula | Formula | Formula | Formula | Formula |
| | 61 | 62 | 63 | 64 | 65 |
| Quaternary ammonium | 0.01 to 0.3 | 0.01 to 0.3 | 0.02 to 0.2 | 0.02 to 0.2 | 0.05 to 0.1 |
| compound | | | | | |
| Ethylhexyl glycerol | 0.1 to 3.0 | 0.15 to 2.0 | 0.15 to 2.0 | 0.2 to 1.0 | 0.2 to 1.0 |
| Bactericide | 0.1 to 2.0 | 0.1 to 2.0 | 0.2 to 1.6 | 0.2 to 1.6 | 0.5 to 1.4 |
| Non-ionic emulsifier* | 3.0 to 12 | 3.0 to 12 | 4.0 to 10 | 4.0 to 10 | 5.0 to 8.0 |
| Water, optional | up to 100 | up to 100 | up to 100 | up to 100 | up to 100 |
| additive | | | | | |

*Steareth-2, steareth-21 or their combinations

| | Formula | Formula | Formula | Formula | Formula |
|---|---|---|---|---|---|
| | 66 | 67 | 68 | 69 | 70 |
| Benzalkonium chloride | 0.01 to 0.3 | 0.01 to 0.3 | 0.02 to 0.2 | 0.02 to 0.2 | 0.05 to 0.1 |
| Ethylhexyl glycerol | 0.1 to 3.0 | 0.15 to 2.0 | 0.15 to 2.0 | 0.2 to 1.0 | 0.2 to 1.0 |
| Bactericide | 0.1 to 2.0 | 0.1 to 2.0 | 0.2 to 1.6 | 0.2 to 1.6 | 0.5 to 1.4 |
| Non-ionic emulsifier* | 3.0 to 12 | 3.0 to 12 | 4.0 to 10 | 4.0 to 10 | 5.0 to 8.0 |
| Water, optional | up to 100 | up to 100 | up to 100 | up to 100 | up to 100 |
| additive | | | | | |

*Steareth-2, steareth-21 or their combinations

| | Formula | Formula | Formula | Formula | Formula |
|---|---|---|---|---|---|
| | 71 | 72 | 73 | 74 | 75 |
| Quaternary ammonium | 0.01 to 0.3 | 0.01 to 0.3 | 0.02 to 0.2 | 0.02 to 0.2 | 0.05 to 0.1 |
| compound | | | | | |
| Ethylhexyl glycerol | 0.1 to 3.0 | 0.15 to 2.0 | 0.15 to 2.0 | 0.2 to 1.0 | 0.2 to 1.0 |
| Phenoxy ethanol | 0.1 to 2.0 | 0.1 to 2.0 | 0.2 to 1.6 | 0.2 to 1.6 | 0.5 to 1.4 |
| Non-ionic emulsifier | 3.0 to 12 | 3.0 to 12 | 4.0 to 10 | 4.0 to 10 | 5.0 to 8.0 |
| Water, optional | up to 100 | up to 100 | up to 100 | up to 100 | up to 100 |
| additive | | | | | |
| | Formula | Formula | Formula | Formula | Formula |
| | 76 | 77 | 78 | 79 | 80 |
| Benzalkonium chloride | 0.01 to 0.3 | 0.01 to 0.3 | 0.02 to 0.2 | 0.02 to 0.2 | 0.05 to 0.1 |
| Ethylhexyl glycerol | 0.1 to 3.0 | 0.15 to 2.0 | 0.15 to 2.0 | 0.2 to 1.0 | 0.2 to 1.0 |
| Phenoxy ethanol | 0.1 to 2.0 | 0.1 to 2.0 | 0.2 to 1.6 | 0.2 to 1.6 | 0.5 to 1.4 |
| Non-ionic emulsifier | 3.0 to 12 | 3.0 to 12 | 4.0 to 10 | 4.0 to 10 | 5.0 to 8.0 |
| Water, optional | up to 100 | up to 100 | up to 100 | up to 100 | up to 100 |
| additive | | | | | |
| | Formula | Formula | Formula | Formula | Formula |
| | 81 | 82 | 83 | 84 | 85 |
| Quaternary ammonium | 0.01 to 0.3 | 0.01 to 0.3 | 0.02 to 0.2 | 0.02 to 0.2 | 0.05 to 0.1 |
| compound | | | | | |
| Ethylhexyl glycerol | 0.1 to 3.0 | 0.15 to 2.0 | 0.15 to 2.0 | 0.2 to 1.0 | 0.2 to 1.0 |
| Phenoxy ethanol | 0.1 to 2.0 | 0.1 to 2.0 | 0.2 to 1.6 | 0.2 to 1.6 | 0.5 to 1.4 |
| Non-ionic emulsifier* | 3.0 to 12 | 3.0 to 12 | 4.0 to 10 | 4.0 to 10 | 5.0 to 8.0 |
| Water, optional | up to 100 | up to 100 | up to 100 | up to 100 | up to 100 |
| additive | | | | | |

*Steareth-2, steareth-21 or their combinations

| | Formula | Formula | Formula | Formula | Formula |
|---|---|---|---|---|---|
| | 86 | 87 | 88 | 89 | 90 |
| Benzalkonium chloride | 0.01 to 0.3 | 0.01 to 0.3 | 0.02 to 0.2 | 0.02 to 0.2 | 0.05 to 0.1 |
| Ethylhexyl glycerol | 0.1 to 3.0 | 0.15 to 2.0 | 0.15 to 2.0 | 0.2 to 1.0 | 0.2 to 1.0 |
| Phenoxy ethanol | 0.1 to 2.0 | 0.1 to 2.0 | 0.2 to 1.6 | 0.2 to 1.6 | 0.5 to 1.4 |
| Non-ionic emulsifier* | 3.0 to 12 | 3.0 to 12 | 4.0 to 10 | 4.0 to 10 | 5.0 to 8.0 |
| Water, optional | up to 100 | up to 100 | up to 100 | up to 100 | up to 100 |
| additive | | | | | |

*Steareth-2, steareth-21 or their combinations

| | Formula | Formula | Formula | Formula | Formula |
|---|---|---|---|---|---|
| | 101 | 102 | 103 | 104 | 105 |
| Quaternary ammonium | 0.01 to 0.3 | 0.01 to 0.3 | 0.02 to 0.2 | 0.02 to 0.2 | 0.05 to 0.1 |
| compound | | | | | |
| Ethylhexyl glycerol | 0.1 to 3.0 | 0.15 to 2.0 | 0.15 to 2.0 | 0.2 to 1.0 | 0.2 to 1.0 |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |
| | Formula | Formula | Formula | Formula | Formula |
| | 106 | 107 | 108 | 109 | 110 |
| Benzalkonium chloride | 0.01 to 0.3 | 0.01 to 0.3 | 0.02 to 0.2 | 0.02 to 0.2 | 0.05 to 0.1 |
| Ethylhexyl glycerol | 0.1 to 3.0 | 0.15 to 2.0 | 0.15 to 2.0 | 0.2 to 1.0 | 0.2 to 1.0 |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |
| | Formula | Formula | Formula | Formula | Formula |
| | 111 | 112 | 113 | 114 | 115 |
| Quaternary ammonium | 0.01 to 0.3 | 0.01 to 0.3 | 0.02 to 0.2 | 0.02 to 0.2 | 0.05 to 0.1 |
| compound | | | | | |
| Ethylhexyl glycerol | 0.1 to 3.0 | 0.15 to 2.0 | 0.15 to 2.0 | 0.2 to 1.0 | 0.2 to 1.0 |
| Bactericide | 0.1 to 2.0 | 0.1 to 2.0 | 0.2 to 1.6 | 0.2 to 1.6 | 0.5 to 1.4 |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |
| | Formula | Formula | Formula | Formula | Formula |
| | 116 | 117 | 118 | 119 | 120 |
| Benzalkonium chloride | 0.01 to 0.3 | 0.01 to 0.3 | 0.02 to 0.2 | 0.02 to 0.2 | 0.05 to 0.1 |
| Ethylhexyl glycerol | 0.1 to 3.0 | 0.15 to 2.0 | 0.15 to 2.0 | 0.2 to 1.0 | 0.2 to 1.0 |
| Bactericide | 0.1 to 2.0 | 0.1 to 2.0 | 0.2 to 1.6 | 0.2 to 1.6 | 0.5 to 1.4 |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |
| | Formula | Formula | Formula | Formula | Formula |
| | 121 | 122 | 123 | 124 | 125 |
| Quaternary ammonium | 0.01 to 0.3 | 0.01 to 0.3 | 0.02 to 0.2 | 0.02 to 0.2 | 0.05 to 0.1 |
| compound | | | | | |
| Ethylhexyl glycerol | 0.1 to 3.0 | 0.15 to 2.0 | 0.15 to 2.0 | 0.2 to 1.0 | 0.2 to 1.0 |
| Phenoxy ethanol | 0.1 to 2.0 | 0.1 to 2.0 | 0.2 to 1.6 | 0.2 to 1.6 | 0.5 to 1.4 |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |
| | Formula | Formula | Formula | Formula | Formula |
| | 126 | 127 | 128 | 129 | 130 |
| Benzalkonium chloride | 0.01 to 0.3 | 0.01 to 0.3 | 0.02 to 0.2 | 0.02 to 0.2 | 0.05 to 0.1 |
| Ethylhexyl glycerol | 0.1 to 3.0 | 0.15 to 2.0 | 0.15 to 2.0 | 0.2 to 1.0 | 0.2 to 1.0 |
| Phenoxy ethanol | 0.1 to 2.0 | 0.1 to 2.0 | 0.2 to 1.6 | 0.2 to 1.6 | 0.5 to 1.4 |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |
| | Formula | Formula | Formula | Formula | Formula |
| | 131 | 132 | 133 | 134 | 135 |
| Quaternary ammonium | 0.01 to 0.3 | 0.01 to 0.3 | 0.02 to 0.2 | 0.02 to 0.2 | 0.05 to 0.1 |
| compound | | | | | |
| Ethylhexyl glycerol | 0.1 to 3.0 | 0.15 to 2.0 | 0.15 to 2.0 | 0.2 to 1.0 | 0.2 to 1.0 |
| Non-ionic emulsifier | 3.0 to 12 | 3.0 to 12 | 4.0 to 10 | 4.0 to 10 | 5.0 to 8.0 |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |
| | Formula | Formula | Formula | Formula | Formula |
| | 131A | 132B | 133C | 134D | 135E |
| Quaternary ammonium | 0.01 to 0.3 | 0.01 to 0.3 | 0.02 to 0.2 | 0.02 to 0.2 | 0.05 to 0.1 |
| compound | | | | | |
| Ethylhexyl glycerol | 0.1 to 3.0 | 0.15 to 2.0 | 0.15 to 2.0 | 0.2 to 1.0 | 0.2 to 1.0 |
| Non-ionic emulsifier | 3.0 to 12 | 3.0 to 12 | 4.0 to 10 | 4.0 to 10 | 5.0 to 8.0 |
| Liquid oil (20° C., | 0.1 to 15 | 0.2 to 10 | 0.2 to 10 | 0.5 to 5.0 | 0.5 to 5.0 |
| 1.013 hPa) | | | | | |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |
| | Formula | Formula | Formula | Formula | Formula |
| | 136 | 137 | 138 | 139 | 140 |
| Benzalkonium chloride | 0.01 to 0.3 | 0.01 to 0.3 | 0.02 to 0.2 | 0.02 to 0.2 | 0.05 to 0.1 |
| Ethylhexyl glycerol | 0.1 to 3.0 | 0.15 to 2.0 | 0.15 to 2.0 | 0.2 to 1.0 | 0.2 to 1.0 |
| Non-ionic emulsifier | 3.0 to 12 | 3.0 to 12 | 4.0 to 10 | 4.0 to 10 | 5.0 to 8.0 |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |
| | Formula | Formula | Formula | Formula | Formula |
| | 136A | 137B | 138C | 139D | 140E |
| Benzalkonium chloride | 0.01 to 0.3 | 0.01 to 0.3 | 0.02 to 0.2 | 0.02 to 0.2 | 0.05 to 0.1 |
| Ethylhexyl glycerol | 0.1 to 3.0 | 0.15 to 2.0 | 0.15 to 2.0 | 0.2 to 1.0 | 0.2 to 1.0 |
| Non-ionic emulsifier | 3.0 to 12 | 3.0 to 12 | 4.0 to 10 | 4.0 to 10 | 5.0 to 8.0 |
| Liquid oil (20° C., | 0.1 to 15 | 0.2 to 10 | 0.2 to 10 | 0.5 to 5.0 | 0.5 to 5.0 |
| 1.013 hPa) | | | | | |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |
| | Formula | Formula | Formula | Formula | Formula |
| | 141 | 142 | 143 | 144 | 145 |
| Quaternary ammonium | 0.01 to 0.3 | 0.01 to 0.3 | 0.02 to 0.2 | 0.02 to 0.2 | 0.05 to 0.1 |
| compound | | | | | |
| Ethylhexyl glycerol | 0.1 to 3.0 | 0.15 to 2.0 | 0.15 to 2.0 | 0.2 to 1.0 | 0.2 to 1.0 |
| Non-ionic emulsifier* | 3.0 to 12 | 3.0 to 12 | 4.0 to 10 | 4.0 to 10 | 5.0 to 8.0 |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |

*Steareth-2, steareth-21 or their combinations

| | Formula | Formula | Formula | Formula | Formula |
|---|---|---|---|---|---|
| | 141A | 142B | 143C | 144D | 145E |
| Quaternary ammonium | 0.01 to 0.3 | 0.01 to 0.3 | 0.02 to 0.2 | 0.02 to 0.2 | 0.05 to 0.1 |
| compound | | | | | |
| Ethylhexyl glycerol | 0.1 to 3.0 | 0.15 to 2.0 | 0.15 to 2.0 | 0.2 to 1.0 | 0.2 to 1.0 |
| Non-ionic emulsifier* | 3.0 to 12 | 3.0 to 12 | 4.0 to 10 | 4.0 to 10 | 5.0 to 8.0 |
| Liquid oil (20° C., | 0.1 to 15 | 0.2 to 10 | 0.2 to 10 | 0.5 to 5.0 | 0.5 to 5.0 |
| 1.013 hPa) | | | | | |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |

*Steareth-2, steareth-21 or their combinations

| | Formula | Formula | Formula | Formula | Formula |
|---|---|---|---|---|---|
| | 146 | 147 | 148 | 149 | 150 |
| Benzalkonium chloride | 0.01 to 0.3 | 0.01 to 0.3 | 0.02 to 0.2 | 0.02 to 0.2 | 0.05 to 0.1 |
| Ethylhexyl glycerol | 0.1 to 3.0 | 0.15 to 2.0 | 0.15 to 2.0 | 0.2 to 1.0 | 0.2 to 1.0 |
| Non-ionic emulsifier* | 3.0 to 12 | 3.0 to 12 | 4.0 to 10 | 4.0 to 10 | 5.0 to 8.0 |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |

*Steareth-2, steareth-21 or their combinations

| | Formula | Formula | Formula | Formula | Formula |
|---|---|---|---|---|---|
| | 146A | 147B | 148C | 149D | 150E |
| Benzalkonium chloride | 0.01 to 0.3 | 0.01 to 0.3 | 0.02 to 0.2 | 0.02 to 0.2 | 0.05 to 0.1 |
| Ethylhexyl glycerol | 0.1 to 3.0 | 0.15 to 2.0 | 0.15 to 2.0 | 0.2 to 1.0 | 0.2 to 1.0 |
| Non-ionic emulsifier* | 3.0 to 12 | 3.0 to 12 | 4.0 to 10 | 4.0 to 10 | 5.0 to 8.0 |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |

*Steareth-2, steareth-21 or combinations thereof

| | Formula | Formula | Formula | Formula | Formula |
|---|---|---|---|---|---|
| | 151 | 152 | 153 | 154 | 155 |
| Benzalkonium chloride | 0.01 to 0.3 | 0.01 to 0.3 | 0.02 to 0.2 | 0.02 to 0.2 | 0.05 to 0.1 |
| Ethylhexyl glycerol | 0.1 to 3.0 | 0.15 to 2.0 | 0.15 to 2.0 | 0.2 to 1.0 | 0.2 to 1.0 |
| Non-ionic emulsifier* | 3.0 to 12 | 3.0 to 12 | 4.0 to 10 | 4.0 to 10 | 5.0 to 8.0 |
| Liquid oil (20° C., | 0.1 to 15 | 0.2 to 10 | 0.2 to 10 | 0.5 to 5.0 | 0.5 to 5.0 |
| 1.013 hPa) | | | | | |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |

*Steareth-2, steareth-21 or their combinations

| | Formula | Formula | Formula | Formula | Formula |
|---|---|---|---|---|---|
| | 156 | 157 | 158 | 159 | 160 |
| Quaternary ammonium | 0.01 to 0.3 | 0.01 to 0.3 | 0.02 to 0.2 | 0.02 to 0.2 | 0.05 to 0.1 |
| compound | | | | | |
| Ethylhexyl glycerol | 0.1 to 3.0 | 0.15 to 2.0 | 0.15 to 2.0 | 0.2 to 1.0 | 0.2 to 1.0 |
| Bactericide | 0.1 to 2.0 | 0.1 to 2.0 | 0.2 to 1.6 | 0.2 to 1.6 | 0.5 to 1.4 |
| Non-ionic emulsifier | 3.0 to 12 | 3.0 to 12 | 4.0 to 10 | 4.0 to 10 | 5.0 to 8.0 |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |
| | Formula | Formula | Formula | Formula | Formula |
| | 161 | 162 | 163 | 164 | 165 |
| Quaternary ammonium | 0.01 to 0.3 | 0.01 to 0.3 | 0.02 to 0.2 | 0.02 to 0.2 | 0.05 to 0.1 |
| compound | | | | | |
| Ethylhexyl glycerol | 0.1 to 3.0 | 0.15 to 2.0 | 0.15 to 2.0 | 0.2 to 1.0 | 0.2 to 1.0 |
| Bactericide | 0.1 to 2.0 | 0.1 to 2.0 | 0.2 to 1.6 | 0.2 to 1.6 | 0.5 to 1.4 |
| Non-ionic emulsifier | 3.0 to 12 | 3.0 to 12 | 4.0 to 10 | 4.0 to 10 | 5.0 to 8.0 |
| Liquid oil (20° C., | 0.1 to 15 | 0.2 to 10 | 0.2 to 10 | 0.5 to 5.0 | 0.5 to 5.0 |
| 1.013 hPa) | | | | | |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |
| | Formula | Formula | Formula | Formula | Formula |
| | 166 | 167 | 168 | 169 | 170 |
| Benzalkonium chloride | 0.01 to 0.3 | 0.01 to 0.3 | 0.02 to 0.2 | 0.02 to 0.2 | 0.05 to 0.1 |
| Ethylhexyl glycerol | 0.1 to 3.0 | 0.15 to 2.0 | 0.15 to 2.0 | 0.2 to 1.0 | 0.2 to 1.0 |
| Bactericide | 0.1 to 2.0 | 0.1 to 2.0 | 0.2 to 1.6 | 0.2 to 1.6 | 0.5 to 1.4 |
| Non-ionic emulsifier | 3.0 to 12 | 3.0 to 12 | 4.0 to 10 | 4.0 to 10 | 5.0 to 8.0 |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |
| | Formula | Formula | Formula | Formula | Formula |
| | 171 | 172 | 173 | 174 | 175 |
| Benzalkonium chloride | 0.01 to 0.3 | 0.01 to 0.3 | 0.02 to 0.2 | 0.02 to 0.2 | 0.05 to 0.1 |
| Ethylhexyl glycerol | 0.1 to 3.0 | 0.15 to 2.0 | 0.15 to 2.0 | 0.2 to 1.0 | 0.2 to 1.0 |
| Bactericide | 0.1 to 2.0 | 0.1 to 2.0 | 0.2 to 1.6 | 0.2 to 1.6 | 0.5 to 1.4 |
| Non-ionic emulsifier | 3.0 to 12 | 3.0 to 12 | 4.0 to 10 | 4.0 to 10 | 5.0 to 8.0 |
| Liquid oil (20° C., | 0.1 to 15 | 0.2 to 10 | 0.2 to 10 | 0.5 to 5.0 | 0.5 to 5.0 |
| 1.013 hPa) | | | | | |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |
| | Formula | Formula | Formula | Formula | Formula |
| | 176 | 177 | 178 | 179 | 180 |
| Quaternary ammonium | 0.01 to 0.3 | 0.01 to 0.3 | 0.02 to 0.2 | 0.02 to 0.2 | 0.05 to 0.1 |
| compound | | | | | |
| Ethylhexyl glycerol | 0.1 to 3.0 | 0.15 to 2.0 | 0.15 to 2.0 | 0.2 to 1.0 | 0.2 to 1.0 |
| Bactericide | 0.1 to 2.0 | 0.1 to 2.0 | 0.2 to 1.6 | 0.2 to 1.6 | 0.5 to 1.4 |
| Non-ionic emulsifier* | 3.0 to 12 | 3.0 to 12 | 4.0 to 10 | 4.0 to 10 | 5.0 to 8.0 |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |

*Steareth-2, steareth-21 or their combinations

| | Formula | Formula | Formula | Formula | Formula |
|---|---|---|---|---|---|
| | 181 | 182 | 183 | 184 | 185 |
| Quaternary ammonium | 0.01 to 0.3 | 0.01 to 0.3 | 0.02 to 0.2 | 0.02 to 0.2 | 0.05 to 0.1 |
| compound | | | | | |
| Ethylhexyl glycerol | 0.1 to 3.0 | 0.15 to 2.0 | 0.15 to 2.0 | 0.2 to 1.0 | 0.2 to 1.0 |
| Bactericide | 0.1 to 2.0 | 0.1 to 2.0 | 0.2 to 1.6 | 0.2 to 1.6 | 0.5 to 1.4 |
| Non-ionic emulsifier* | 3.0 to 12 | 3.0 to 12 | 4.0 to 10 | 4.0 to 10 | 5.0 to 8.0 |
| Liquid oil (20° C., | 0.1 to 15 | 0.2 to 10 | 0.2 to 10 | 0.5 to 5.0 | 0.5 to 5.0 |
| 1.013 hPa) | | | | | |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |

*Steareth-2, steareth-21 or their combinations

| | Formula | Formula | Formula | Formula | Formula |
|---|---|---|---|---|---|
| | 186 | 187 | 188 | 189 | 190 |
| Benzalkonium chloride | 0.01 to 0.3 | 0.01 to 0.3 | 0.02 to 0.2 | 0.02 to 0.2 | 0.05 to 0.1 |
| Ethylhexyl glycerol | 0.1 to 3.0 | 0.15 to 2.0 | 0.15 to 2.0 | 0.2 to 1.0 | 0.2 to 1.0 |
| Bactericide | 0.1 to 2.0 | 0.1 to 2.0 | 0.2 to 1.6 | 0.2 to 1.6 | 0.5 to 1.4 |
| Non-ionic emulsifier* | 3.0 to 12 | 3.0 to 12 | 4.0 to 10 | 4.0 to 10 | 5.0 to 8.0 |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |

*Steareth-2, steareth-21 or their combinations

| | Formula | Formula | Formula | Formula | Formula |
|---|---|---|---|---|---|
| | 191 | 192 | 193 | 194 | 195 |
| Benzalkonium chloride | 0.01 to 0.3 | 0.01 to 0.3 | 0.02 to 0.2 | 0.02 to 0.2 | 0.05 to 0.1 |
| Ethylhexyl glycerol | 0.1 to 3.0 | 0.15 to 2.0 | 0.15 to 2.0 | 0.2 to 1.0 | 0.2 to 1.0 |
| Bactericide | 0.1 to 2.0 | 0.1 to 2.0 | 0.2 to 1.6 | 0.2 to 1.6 | 0.5 to 1.4 |
| Non-ionic emulsifier* | 3.0 to 12 | 3.0 to 12 | 4.0 to 10 | 4.0 to 10 | 5.0 to 8.0 |
| Liquid oil (20° C., | 0.1 to 15 | 0.2 to 10 | 0.2 to 10 | 0.5 to 5.0 | 0.5 to 5.0 |
| 1.013 hPa) | | | | | |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |

*Steareth-2, steareth-21 or their combinations

| | Formula | Formula | Formula | Formula | Formula |
|---|---|---|---|---|---|
| | 196 | 197 | 198 | 199 | 200 |
| Quaternary ammonium | 0.01 to 0.3 | 0.01 to 0.3 | 0.02 to 0.2 | 0.02 to 0.2 | 0.05 to 0.1 |
| compound | | | | | |
| Ethylhexyl glycerol | 0.1 to 3.0 | 0.15 to 2.0 | 0.15 to 2.0 | 0.2 to 1.0 | 0.2 to 1.0 |
| Phenoxy ethanol | 0.1 to 2.0 | 0.1 to 2.0 | 0.2 to 1.6 | 0.2 to 1.6 | 0.5 to 1.4 |
| Non-ionic emulsifier | 3.0 to 12 | 3.0 to 12 | 4.0 to 10 | 4.0 to 10 | 5.0 to 8.0 |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |
| | Formula | Formula | Formula | Formula | Formula |
| | 201 | 202 | 203 | 204 | 205 |
| Quaternary ammonium | 0.01 to 0.3 | 0.01 to 0.3 | 0.02 to 0.2 | 0.02 to 0.2 | 0.05 to 0.1 |
| compound | | | | | |
| Ethylhexyl glycerol | 0.1 to 3.0 | 0.15 to 2.0 | 0.15 to 2.0 | 0.2 to 1.0 | 0.2 to 1.0 |
| Phenoxy ethanol | 0.1 to 2.0 | 0.1 to 2.0 | 0.2 to 1.6 | 0.2 to 1.6 | 0.5 to 1.4 |
| Non-ionic emulsifier | 3.0 to 12 | 3.0 to 12 | 4.0 to 10 | 4.0 to 10 | 5.0 to 8.0 |
| Liquid oil (20° C., | 0.1 to 15 | 0.2 to 10 | 0.2 to 10 | 0.5 to 5.0 | 0.5 to 5.0 |
| 1.013 hPa) | | | | | |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |
| | Formula | Formula | Formula | Formula | Formula |
| | 206 | 207 | 208 | 209 | 210 |
| Benzalkonium chloride | 0.01 to 0.3 | 0.01 to 0.3 | 0.02 to 0.2 | 0.02 to 0.2 | 0.05 to 0.1 |
| Ethylhexyl glycerol | 0.1 to 3.0 | 0.15 to 2.0 | 0.15 to 2.0 | 0.2 to 1.0 | 0.2 to 1.0 |
| Phenoxy ethanol | 0.1 to 2.0 | 0.1 to 2.0 | 0.2 to 1.6 | 0.2 to 1.6 | 0.5 to 1.4 |
| Non-ionic emulsifier | 3.0 to 12 | 3.0 to 12 | 4.0 to 10 | 4.0 to 10 | 5.0 to 8.0 |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |
| | Formula | Formula | Formula | Formula | Formula |
| | 211 | 212 | 213 | 214 | 215 |
| Benzalkonium chloride | 0.01 to 0.3 | 0.01 to 0.3 | 0.02 to 0.2 | 0.02 to 0.2 | 0.05 to 0.1 |
| Ethylhexyl glycerol | 0.1 to 3.0 | 0.15 to 2.0 | 0.15 to 2.0 | 0.2 to 1.0 | 0.2 to 1.0 |
| Phenoxy ethanol | 0.1 to 2.0 | 0.1 to 2.0 | 0.2 to 1.6 | 0.2 to 1.6 | 0.5 to 1.4 |
| Non-ionic emulsifier | 3.0 to 12 | 3.0 to 12 | 4.0 to 10 | 4.0 to 10 | 5.0 to 8.0 |
| Liquid oil (20° C., | 0.1 to 15 | 0.2 to 10 | 0.2 to 10 | 0.5 to 5.0 | 0.5 to 5.0 |
| 1.013 hPa) | | | | | |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |
| | Formula | Formula | Formula | Formula | Formula |
| | 216 | 217 | 218 | 219 | 220 |
| Quaternary ammonium | 0.01 to 0.3 | 0.01 to 0.3 | 0.02 to 0.2 | 0.02 to 0.2 | 0.05 to 0.1 |
| compound | | | | | |
| Ethylhexyl glycerol | 0.1 to 3.0 | 0.15 to 2.0 | 0.15 to 2.0 | 0.2 to 1.0 | 0.2 to 1.0 |
| Phenoxy ethanol | 0.1 to 2.0 | 0.1 to 2.0 | 0.2 to 1.6 | 0.2 to 1.6 | 0.5 to 1.4 |
| Non-ionic emulsifier* | 3.0 to 12 | 3.0 to 12 | 4.0 to 10 | 4.0 to 10 | 5.0 to 8.0 |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |

*Steareth-2, steareth-21 or their combinations

| | Formula | Formula | Formula | Formula | Formula |
|---|---|---|---|---|---|
| | 221 | 222 | 223 | 224 | 225 |
| Quaternary ammonium | 0.01 to 0.3 | 0.01 to 0.3 | 0.02 to 0.2 | 0.02 to 0.2 | 0.05 to 0.1 |
| compound | | | | | |
| Ethylhexyl glycerol | 0.1 to 3.0 | 0.15 to 2.0 | 0.15 to 2.0 | 0.2 to 1.0 | 0.2 to 1.0 |
| Phenoxy ethanol | 0.1 to 2.0 | 0.1 to 2.0 | 0.2 to 1.6 | 0.2 to 1.6 | 0.5 to 1.4 |
| Non-ionic emulsifier* | 3.0 to 12 | 3.0 to 12 | 4.0 to 10 | 4.0 to 10 | 5.0 to 8.0 |
| Liquid oil (20° C., | 0.1 to 15 | 0.2 to 10 | 0.2 to 10 | 0.5 to 5.0 | 0.5 to 5.0 |
| 1.013 hPa) | | | | | |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |

*Steareth-2, steareth-21 or their combinations

| | Formula | Formula | Formula | Formula | Formula |
|---|---|---|---|---|---|
| | 226 | 227 | 228 | 229 | 230 |
| Benzalkonium chloride | 0.01 to 0.3 | 0.01 to 0.3 | 0.02 to 0.2 | 0.02 to 0.2 | 0.05 to 0.1 |
| Ethylhexyl glycerol | 0.1 to 3.0 | 0.15 to 2.0 | 0.15 to 2.0 | 0.2 to 1.0 | 0.2 to 1.0 |
| Phenoxy ethanol | 0.1 to 2.0 | 0.1 to 2.0 | 0.2 to 1.6 | 0.2 to 1.6 | 0.5 to 1.4 |
| Non-ionic emulsifier* | 3.0 to 12 | 3.0 to 12 | 4.0 to 10 | 4.0 to 10 | 5.0 to 8.0 |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |

*Steareth-2, steareth-21 or their combinations

| | Formula | Formula | Formula | Formula | Formula |
|---|---|---|---|---|---|
| | 231 | 232 | 233 | 234 | 235 |
| Benzalkonium chloride | 0.01 to 0.3 | 0.01 to 0.3 | 0.02 to 0.2 | 0.02 to 0.2 | 0.05 to 0.1 |
| Ethylhexyl glycerol | 0.1 to 3.0 | 0.15 to 2.0 | 0.15 to 2.0 | 0.2 to 1.0 | 0.2 to 1.0 |
| Phenoxy ethanol | 0.1 to 2.0 | 0.1 to 2.0 | 0.2 to 1.6 | 0.2 to 1.6 | 0.5 to 1.4 |
| Non-ionic emulsifier* | 3.0 to 12 | 3.0 to 12 | 4.0 to 10 | 4.0 to 10 | 5.0 to 8.0 |
| Liquid oil (20° C., | 0.1 to 15 | 0.2 to 10 | 0.2 to 10 | 0.5 to 5.0 | 0.5 to 5.0 |
| 1.013 hPa) | | | | | |
| Ethanol | <5.0 | <1.0 | <1.0 | <1.0 | <0.1 |
| Water | 75 to 96 | 80 to 96 | 85 to 94 | 85 to 94 | 85 to 94 |
| Optional additives | up to 100 | up to 100 | up to 100 | up to 100 | up to 100 |

*Steareth-2, steareth-21 or their combinations

The deodorizing cosmetic agent can be applied by means of various methods. According to a preferred embodiment, the perspiration-inhibiting cosmetic agent can be packaged as a spray application. The spray is applied with a spraying device which comprises a container filled with deodorizing cosmetic agent. The filling may be pressurized with a propellant (compressed gas cans, aerosol dispensers), or can be a mechanically-operated pump nebulizer without a propellant (pump sprays/squeeze bottles). Preferably, propellant is dispensed with, meaning that preferred agents are therefore free from propellant. The resulting preferred propellant-free agents are applied preferably by means of a pump nebulizer. A pump nebulizer containing a deodorizing cosmetic agent according to the invention is therefore a further subject matter of the present invention.

The deodorizing agent can furthermore thus also be packaged as a roll-on. Such a packaging is particularly preferred, alongside packaging as a pump nebulizer. This can for example be applied with a tracking ball. Such items have a sphere housed in a ball bed, which sphere can be moved by movement over a surface. In so doing, the sphere receives some of the perspiration-inhibiting cosmetic agent according to the invention to be distributed and conveys this to the surface to be treated. Roll-on applicators containing a deodorizing cosmetic agent according to the invention are preferred.

A combination with a disposable substrate, for example a substrate selected from the group of wipes, pads and wads, is suitable as an alternative form of application of the deodorizing cosmetic agents. Wet wipes, i.e. wet wipes which are pre-packaged, preferably individually packaged, for the user, as are well known e.g. in the field of glass cleaning or in the field of moist toilet papers, are particularly preferred. Such wet wipes, which advantageously can also include preservatives, are impregnated or loaded with a deodorizing cosmetic agent according to the invention, and preferably packaged individually. They can e.g. be used as deodorant wipes, which is of particular interest for use when in transit. Preferred substrate materials are selected from porous, flat wipes. They can consist of a fibrous or cellular flexible material which has sufficient mechanical stability and simultaneously softness for application to the skin. These wipes include wipes made of woven and unwoven synthetic and natural fibers, felt, paper or cellular material, such as hydrophilic polyurethane foam. According to the invention, preferred deodorizing or perspiration-inhibiting substrates can be obtained by impregnating or also by melting onto a substrate a perspiration-inhibiting cosmetic agent according to the invention.

A further subject matter of the claims is a non-therapeutic cosmetic method for preventing and/or reducing body odor, in which a cosmetic agent according to the invention is applied to the skin, in particular to the skin of the armpits, and remains on the skin for at least 1 hour, preferably for at least 2 hours, preferably for at least 4 hours, in particular for at least 6 hours.

As stated at the outset, the cosmetic agents according to the invention have an advantageous influence on the onset of body odor. The use of these cosmetic agents for preventing and/or reducing body odor is a further subject matter of the present invention.

### EXAMPLES

The following deodorant emulsions were produced (details in wt.-%):

| | 1 | 2 | 3 | 4 | 5 | 6 |
|---|---|---|---|---|---|---|
| PPG-15 Stearyl ether | 0.5 | 0.5 | 0.5 | 0.5 | 0.5 | 0.5 |
| Steareth-2 | 3.6 | 3.6 | 3.6 | 3.6 | 3.6 | 3.6 |
| Steareth-21 | 2.2 | 2.2 | 2.2 | 2.2 | 2.2 | 2.2 |
| Ethylhexyl glycerol | 0.5 | 0.2 | 0.9 | — | 0.9 | — |
| Barquat¹⁾ | 0.2 | 0.2 | 0.1 | 0.2 | — | — |
| Citric acid triethyl ester | — | — | — | — | — | 2.0 |
| Phenoxy ethanol | 0.9 | 0.9 | 0.9 | 0.9 | 0.9 | 0.9 |
| EDTA | 0.1 | 0.1 | 0.1 | 0.1 | 0.1 | 0.1 |
| Perfume | 1.0 | 1.0 | 1.0 | 1.0 | 1.0 | 1.0 |
| Water | up to | up to | up to | up to | up to | up to 100 |
| | 100 | 100 | 100 | 100 | 100 | |
| Stability (4 weeks, | stable | stable | stable | stable | stable | Phase |
| 40° C.) | | | | | | separation |
| Effect against body odor | + | + | + | − | − | − |

¹⁾INCI: Benzalkonium chloride; 50% (Lonza)

Compositions 1 to 3 according to the invention are characterized by a high storage stability, high microbiological stability and good effect against body odor.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A deodorizing cosmetic agent in the form of an aqueous emulsion comprising:
at least one quaternary ammonium compound and
ethylhexyl glycerol,
wherein the deodorizing cosmetic agent does not include any aluminum-containing compounds.

2. The deodorizing cosmetic agents according to claim 1, wherein the quaternary ammonium compound is selected from the group consisting of behenyl trimonium chloride, cetrimonium bromide, cetrimonium chloride, lauryl trimonium bromide, lauryl trimonium chloride, stearyl trimonium bromide, stearyl trimonium chloride, benzethonium chloride, benzalkonium chloride and mixtures thereof.

3. The deodorizing cosmetic agents according to claim 1, wherein the quaternary ammonium compound includes benzalkonium chloride.

4. The deodorizing cosmetic agent according to claim 1, wherein the agent includes the quaternary ammonium compound in a quantity of 0.01 to 0.3 wt.-%, relative to the total weight of the agent.

5. The deodorizing cosmetic agent according to claim 1, wherein the agent includes the quaternary ammonium compound in a quantity of 0.02 to 0.2 wt.-%, relative to the total weight of the agent.

6. The deodorizing cosmetic agent according to claim 1, wherein the agent includes the quaternary ammonium compound in a quantity of 0.05 to 0.1 wt.-%, relative to the total weight of the agent.

7. The deodorizing cosmetic agent according to claim 1, wherein the agent includes the ethylhexyl glycerol in a quantity of from 0.1 to 3.0 wt.-%, relative to the total weight of the agent.

8. The deodorizing cosmetic agent according to claim 1, wherein the agent includes the ethylhexyl glycerol in a quantity of from 0.15 to 2.0 wt.-%, relative to the total weight of the agent.

9. The deodorizing cosmetic agent according to claim 1, wherein the agent includes the ethylhexyl glycerol in a quantity of from 0.2 to 1.0 wt.-%, relative to the total weight of the agent.

10. The deodorizing cosmetic agent according to claim 1, wherein the agent includes at least one bactericide selected from the group consisting of: chlorhexidine, farnesol, triclosan, chloroxylenol and phenoxy ethanol.

11. The deodorizing cosmetic agent according to claim 1, wherein the agent includes at least one bactericide includes phenoxy ethanol.

12. The deodorizing cosmetic agent according to claim 1, wherein the agent includes ethanol in quantities of less than 5.0 wt.-%, relative to the total weight of the agent.

13. The deodorizing cosmetic agent according to claim 1, wherein the agent includes ethanol in quantities of less than 1.0 wt.-%, relative to the total weight of the agent.

14. The deodorizing cosmetic agent according to claim 1, wherein the agent includes ethanol in quantities of less than 0.1 wt.-%, relative to the total weight of the agent.

15. The deodorizing cosmetic agent according to claim 1, wherein the agent includes water in quantities of 75 to 96 wt.-%, relative to the total weight of the agent.

16. The deodorizing cosmetic agent according to claim 1, wherein the agent includes water in quantities of 80 to 96 wt.-%, relative to the total weight of the agent.

17. The deodorizing cosmetic agent according to claim 1, wherein the agent includes water in quantities of 85 to 95 wt.-%, relative to the total weight of the agent.

18. A non-therapeutic cosmetic method for preventing and/or reducing body odor, comprising applying to skin a cosmetic agent according to claim 1 and leaving the cosmetic agent on the skin for at least 1 hour.

19. The non-therapeutic cosmetic method for preventing and/or reducing body odor, wherein the agent is left on the skin for at least 2 hours.
